(19) United States
(12) Patent Application Publication
(10) Pub. No.: US 2016/0122745 A1
(43) Pub. Date: May 5, 2016

(54) METHOD AND SYSTEM FOR AQUACULTURE OR REDUCING BIOFOULING

(71) Applicants: Andrew Greig JEFFS, Auckland (NZ); JENNI STANLEY, Auckland (NZ); Serena Louise WILKENS, Wellington (NZ); AUCKLAND UNISERVICES LIMITED, Auckland (NZ); NATIONAL INSTITUTE OF WATER AND ATMOSPHERIC RESEARCH LIMITED, Newmarket, Auckland (NZ)

(72) Inventors: Andrew Greig JEFFS, Auckland (NZ); Jenni STANLEY, Auckland (NZ); Serena Louise WILKENS, Wellington (NZ)

(73) Assignees: AUCKLAND UNISERVICES LIMITED, Auckland (NZ); NATIONAL INSTITUTE OF WATER AND ATMOSPHERIC RESEARCH LIMITED, Auckland (NZ)

(21) Appl. No.: 14/889,689

(22) PCT Filed: May 7, 2014
(86) PCT No.: PCT/IB2014/061260

§ 371 Date: Nov. 6, 2015

(30) Foreign Application Priority Data
May 7, 2013 (NZ) 610072

(51) Int. Cl. C12N 13/00; B63B 59/04; A01G 33/00; B08B 17/02; A01K 61/00

(52) U.S. Cl. CPC C12N 13/00; B08B 17/02; A01K 61/005; A01G 33/00; A01K 61/002; B63B 59/04

(57) ABSTRACT

An aquaculture method and a method for reducing biofouling of vessels or submerged structures, the method comprising broadcasting into the marine environment sound at a frequency or in a frequency range effective to attract one or more marine species to the sound source.

### FIELD OF INVENTION

The invention relates to a method and system for use in aquaculture and a method and system for reducing biofouling of vessel hulls or submerged structures or submerged parts thereof.

### BACKGROUND

Marine biofouling is the result of the settlement, growth and colonization of algae and invertebrates on the surface of submerged objects which can create many important and costly problems. One of the most well-known industries plagued by marine fouling organisms since the beginning of its existence is the shipping industry and marine biofouling represents one of their major challenges. Biofouling on ship hulls increases the surface coarseness which, in turn, causes increased frictional resistance leading to a decrease in top speed and range of the ship and an increase in fuel consumption. Millions of dollars are spent each year on attempting to control the fouling on commercial vessels and on the increased fuel costs due to the hydrodynamic drag caused by fouling. Large steel hulled vessels are particularly susceptible to accumulate marine fouling and as a consequence, epibiosis and fouling are extremely common phenomena in the oceans. Vessel biofouling is often characterized by the settlement of invertebrates, however, biofouling communities can range from a fine layer of microscopic algae to a mass of encrusting organisms (e.g. crustaceans, cnidarians, ascidians, bivalves and/or bryozoans).

There are also the wide-ranging implications for biosecurity and vessel-mediated expansion of invasive species in the marine environment. The introduction of non-indigenous species is acknowledged as a major threat to marine biodiversity and a contributor to environmental change.

Various methods of reducing fouling on ships have been proposed and used through the ages which include the use of toxic antifoulant coatings to deter biofoulers or biocides to clear biofouling organisms from the hull surface. Older methods often used copper in multiple chemical forms, however, after a time certain organisms can tolerate the toxicity of copper and can then colonize it thereby shielding other organisms to which copper would otherwise be poisonous. This problem has been partially solved by anti-fouling paints some of which contain toxic biofoulants, such as tributyl tin compounds; however, while they are effective in the short-term, they are very poisonous and may lead to biological degradation and impact non-target species. The impact of these biocides on the environment has led to legislated partial bans and regulation of their use, and there is now extensive research into the discovery and use of low or non-toxic antifoulants to provide better performance and environmental conformity.

In aquaculture a variety of methods are used to promote the settlement of marine species, such as mussels, scallops, clams, crabs and seaweeds. In a hatchery situation this can include the use of chemical and substrate cues which trigger a settlement response of the cultured larvae or spores, often onto settlement substrate that is ideal for practical aquaculture. The collection of wild settlers, seed, or spat, for subsequent grow out in aquaculture systems also often relies on the use of substrate and chemical cues. For example the collection of mussel seed from coastal waters often involves the use of a fibrous rope that provides a substrate which mimics their natural settlement substrate of filamentous seaweed.

### SUMMARY OF INVENTION

In broad terms in one aspect the invention comprises a method of reducing biofouling of a hull or part thereof or any submerged part of a vessel, or of a submerged structure or submerged part of a structure, or a submerged body, which comprises broadcasting into the marine environment in the vicinity of or at the hull, structure, or body sound at a frequency or in a frequency range effective to attract one or more biofouling species to the submerged sound source.

In broad terms in one aspect the invention comprises a method of reducing biofouling of vessels in a port, or of submerged port structures, which comprises broadcasting into the port marine environment sound at a frequency or in a frequency range effective to attract one or more biofouling species to the submerged sound source.

Preferably the method comprises broadcasting into the marine environment sound at a frequency or in a frequency range and/or at a sound intensity and/or which varies, effective to attract one or more biofouling species to the submerged sound source, preferentially away from the submerged hull, structure, or body, preferably to a marine-submersible or submerged sacrificial element associated with the sound source.

In broad terms in another aspect the invention comprises a system or apparatus for reducing biofouling of a hull or part thereof or any submerged part of a vessel, or of a submerged structure or submerged part of a structure, or a submerged body, arranged to broadcast into a marine environment in the vicinity of, or at the hull, structure, or body sound at a frequency or in a frequency range effective to attract one or more biofouling species to the submerged sound source.

In broad terms in a further aspect the invention comprises a system or apparatus for reducing biofouling, which comprises a marine-submersible or submerged sound transducer, a system for driving the transducer to broadcast sound into a marine environment at a frequency or in a frequency range and/or at a sound intensity and/or which varies and which is effective to attract one or more biofouling species towards the submerged sound source, and a marine-submersible or submerged sacrificial element associated with the transducer providing a substrate to which biofouling species may attach.

In broad terms in a further aspect the invention comprises a system or apparatus for reducing biofouling, which comprises a marine-submersible or submerged sound transducer, a system for driving the transducer to broadcast into a marine environment sound at a frequency or in a frequency range and/or at a sound intensity and/or which varies and which is effective to repel or prevent the settlement of one or more biofouling species towards the submerged sound source.

In broad terms in another aspect the invention comprises a system or apparatus for inducing settlement of settlement stages of marine species (such as larvae, post-larvae, propagules, or spores of marine species) desired as seed for subsequent grow out in aquaculture which comprises broadcasting sound into a marine environment or culture vessel in the vicinity of settlement material that can be recovered together with the seed for aquaculture, at a frequency or in a frequency range and/or at a sound intensity and/or which varies and which is effective to attract one or more of the desired aquaculture species to the submerged sound source and settlement material. The system or apparatus comprises a submersible or submerged sound transducer, a system for driving the transducer to broadcast the sound into the marine environment or culture vessel, and a submersible or submerged aquaculture settlement material associated with the transducer providing a substrate to which the settlement stages of marine species may attach.

In broad terms in a further aspect the invention comprises a method for inducing settlement of settlement stages of marine species (such as larvae, post-larvae, propagules, or spores of marine species) desired as seed for subsequent culture for aquaculture which comprises broadcasting sound into a marine environment or culture vessel in the vicinity of settlement material that can be recovered together with the seed for aquaculture, at a frequency or in a frequency range and/or at a sound intensity and/or which varies and which is effective to attract one or more of the desired aquaculture species to the submerged sound source and settlement material.

In various embodiments the method may alternatively or additionally comprise promoting the development, retention, survival and/or growth of settlement stages of marine species (such as larvae, post-larvae, propagules, or spores of marine species). In various embodiments the development, retention, survival and/or growth of settlement stages of marine species may be increased by at least about 1, 5, 10, 15, 20, or 25% or more compared to an untreated control. In one embodiment where the settlement stage is pueruli (crayfish juveniles), the time to moult (TTM) is reduced by at least about 10, 15, 20, 25, 30 or 35% compared to an untreated control.

In various embodiments the method may comprise the step of submerging settlement material specifically adapted for the attachment of the settlement stages of marine species (such as larvae, post-larvae, propagules, or spores of marine species).

In various embodiments the method may further comprise the step of processing the settlement material to recover the seed. In a further embodiment the method may further comprise the step of processing the settlement material once the attached marine species have reached a desired size to recover the marine species.

The induction/promotion of the settlement of the settlement stages with sound may occur in a hatchery (cultured larvae, post-larvae, propagules, or spores in captive conditions) or in coastal waters for the collection of wild seed or spat. The marine species may be for example bivalves such as mussels, scallops, clams, oysters, or cockles, crustaceans such as crabs, lobsters, crayfish, shrimp, or barnacles, and algae such as seaweeds (macro-algae) or microalgae.

In various embodiments the settlement material collects settlement stages of marine species at a rate of more than 1, 5 or 10 individuals per cubic centimetre of settlement material. Useful settlement materials are described below.

In some embodiments the frequency range of the broadcast sound is in or predominantly in the human audible range such as up to 15 kHz, but especially in the range 40 to 1200 Hz, or 40 to 500 Hz. In some embodiments the broadcast sound comprises simple or complex frequencies in and/or over a major part any of the above ranges, including or comprising one or more of short bursts of sounds, or fluctuating intensity of sound at different frequencies, continuous sounds or frequencies, and sounds or frequencies that vary over time regularly and/or randomly. In one embodiment the broadcast sound comprises a repeated recording made from a submerged microphone of real world sound from at least one vessel and/or in a port or natural reef environment.

In some embodiments the sound is broadcast continuously, over one or more days, weeks, months, or years. In other embodiments the sound is broadcast semi-continuously such as during periods of one or more minutes or hours between shorter or longer non-broadcast periods.

Preferably the sound is broadcast in a direction away from the water surface. For example the transducer and/or sacrificial element may be oriented to face away from the water surface.

In some embodiments the sound is broadcast at an intensity of at least about 80, 90, 100, 110 dB or at least 120 dB or more at the source, and useful ranges may be selected between these values (for example, about 80 to about 120 dB).

In this specification:

A ‘hull or part thereof or any submerged part of a vessel’ or similar includes a hull or part thereof of a vessel of any size from a small boat to a larger ocean going vessel, and of any material whether metallic or other, and also includes the submerged part of a propulsion unit of a vessel, and includes the hull or part thereof of a submarine vessel.

A ‘submerged structure or submerged part of a structure’ or similar includes a submerged part or parts of a wharf or pier or other docking or port structure, or of marine equipment operated in a port or other marine environment, or of any other structure in a marine environment such as an oil rig for example.

A ‘a submerged body’ or similar includes any body of any material which is in use submerged such as any commercial fishing equipment which is set submerged for an extended period.

‘Biofouling species’ includes microscopic algae, seaweeds, and “spores” thereof and larger invertebrate organisms such as crustaceans, cnidarians, hydroids, polychaetes, ascidians, bivalves and/or bryozoans.

‘Port’ includes also small marine areas which may comprise only a single short pier or wharf, at which small vessels, such as only recreational vessels, may be berthed, and includes port areas whether defined by man-made structure(s) such as a breakwater or not.

The term “comprising” means “consisting at least in part of”. When interpreting statements in this specification and claims which include the term “comprising”, other features besides the features prefaced by this term in each statement can also be present.

Related terms are to be interpreted similarly.

### BRIEF DESCRIPTION OF THE FIGURES

In the accompanying figures which are referred to further in the subsequent description of experimental work:

FIG. 1 is a spectrogram of: vessel noise recorded from a vessel berthed in port—top line; a High intensity sound treatment—second line; a Low intensity sound treatment—third line; and Silent treatment, i.e., no vessel noise—bottom line;

FIG. 2 is a bar graph showing the percentage mean survival of ascidian larvae for different sound treatments;

FIG. 3 shows the percentage of total number of ascidian larvae swimming over time (h);

FIG. 4 shows the percentage of total number of ascidian larvae metamorphosed;

FIG. 5 is a bar graph showing the mean number of individuals settled of each species for sound and silent treatments; and

FIG. 6 is a non-metric multidimensional scaling (MDS) analysis of total number of organisms attached to settlement panels in sound and silent treatments and with surface and substrate orientations.

FIG. 7 is a settlement response plot showing percentage of all pueruli moulted over time (h) for each experimental sound treatments: silent, kelp-dominated rocky reef, and urchin-dominated rocky reef.

### DETAILED DESCRIPTION

As stated the invention comprises a method of reducing biofouling of a hull or submerged structure or body or part thereof which comprises broadcasting sound into the marine environment in the vicinity of or at the hull, structure, or body but spaced therefrom effective to attract one or more biofouling species to the submerged sound source and typically to a submerged sacrificial element associated with the transducer providing a surface to which biofouling species may attach. This may reduce biofouling by drawing biofouling species away from vessel hulls or submerged structures, and attachment of the biofouling species to the sacrificial element. Preferably one or more biofouling species attach preferentially to the sacrificial element.

Sound may be broadcast into a port marine environment from one or more submerged transducers each with one or more submerged sacrificial elements, to reduce biofouling of vessels in the port, or of a submerged parts of port structures such as wharves or piers, and/or port equipment such as vessel maintenance equipment for example. The transducers and/or sacrificial elements are constructed so as to be able to be left submerged for extended periods such as weeks, months or even years (allowing for raising for periodic maintenance and biofouling removal). The sacrificial elements may be replaced as required after becoming fouled significantly with marine species, or at regular intervals. Two or three or more transducers and/or sacrificial elements may be spaced around a port berthing area.

Alternatively an individual vessel, such as a ship, may have associated with it a submerged sacrificial element attached to but nor forming part of the hull, or comprising a detachable and/or replaceable part of the hull, from at or adjacent which the sound is broadcast at a higher intensity than typical sound from the vessel, to draw biofouling species away from the ship's hull or balance of the hull. In some embodiments transducer and/or sacrificial panel may be deployed only when the vessel is in port.

Additionally, the invention comprises a method for inducing settlement of commercially useful marine species (such as larvae, post-larvae, propagules, or spores of marine species) on a settlement material by broadcasting sound into a marine environment or a culture vessel housing settlement-stages (including, for example, tanks or ponds) in the vicinity of the settlement material. The settlement material may then be processed to collect the marine species for use as seed for subsequent aquaculture into commercially useful forms.

In terms of settlement materials put into water these are a range of materials and structures including fibrous ropes, crushed shell material, cement coated ropes, cement coated plastics, algal coated plastic plates, plastics, cement board materials—these are materials that are preferred for settlement by targeted aquaculture species such as oysters, mussels, clams, abalones, sea urchins, sea cucumbers etc. This material would be deployed in conjunction with sound producing devices into the water, either in tanks or in the field. In so doing the sound attracts the larvae to settle on the dedicated settlement material (settlement structure), and promotes their retention and initial growth, until they reach a size that they are resilient enough to be harvested from the settlement material and transferred to grow out conditions—usually to a farm at sea.

The frequency range of the broadcast sound may be in or predominantly in the human audible range such as up to 15 kHz, or in the range 100 or 200 Hz up to any of to 1, 2 3, 5, 8, 10, or 15 kHz. In some embodiments the broadcast sound comprises broadband simple or complex frequencies in and/or over a major part of any of the above ranges, including or comprising one or more of short bursts of sounds at different frequencies, continuous sounds or frequencies, and sounds or frequencies that vary in over time regularly and/or randomly. In one embodiment the broadcast sound comprises a repeated recording made from a submerged microphone of real world sound from at least one vessel and/or in a port or natural reef environment. In some embodiments the sound is broadcast continuously, over one or more days, weeks, months, or years. In other embodiments the sound is broadcast semi-continuously such as during periods of one or more minutes or hours between shorter or longer non-broadcast periods. In some embodiments the sound is broadcast at an intensity of at least 100 dB re 1 pPa at 1 m or at least 120 dB re 1 pPa at 1 m at the source. Preferably the sound is broadcast in a direction away from the water surface. The transducer and/or sacrificial element and/or settlement material may be oriented to face away from the water surface. The transducer and/or sacrificial element and/or settlement material may separate or the same components. For example a transducer panel may also act as a preferably replaceable sacrificial element or a settlement material. Optionally each transducer may have two or more sacrificial elements or two or move settlement materials associated with it. The sacrificial elements or settlement materials may be in the form of flat panels, of surface area at least about 0.5 m² for example or at least about 1, 2, or 5 m².

### EXPERIMENTAL

The invention is further illustrated by the following description of experimental work:

### Example 1

### Method

Vessel noise recordings: A calibrated hydrophone was used to continuously record 5 minutes of underwater sound emitted by a 126-m long steel-hulled passenger ferry berthed and operating on ship-based generator power supply. No other machinery was operational during the recordings. The hydrophone was placed 3 m from the hull, port side at mid-ship and lowered 3 m into the water, and recordings were repeated 4 times. During the recording phase the output was captured on a calibrated digital recorder. Digital recordings were downloaded onto a PC and the spectral composition and source sound level calculated. A four minute sequence of the recording was transferred onto an MP3 player for playback.

Source of ascidian larvae: Ascidian larvae, Ciona savignyi, were supplied by Cawthron Institute (Nelson, New Zealand). Adult specimens were longitudinally dissected and the sperm and eggs suctioned out using separate glass Pasteur pipettes. C. savignyi are hermaphroditic so eggs and sperm can be removed independently from the gonoducts. Different donor specimens were used to obtain cross fertilisation. The eggs were placed into a petri dish containing 25 ml of sterile seawater and approximately 300 μl of concentrated sperm was added (thereby diluting the sperm and preventing an excess which sticks to the follicular cells of the eggs, endangering insemination). The petri dishes containing eggs and sperm were gently agitated to ensure mixing of gametes. One hour after insemination, the seawater was changed to remove surplus sperm and the petri dishes placed at 18-20° C. for 15-18 hours to allow development. Immediately prior to hatching (which was developmentally confirmed using light microscopy), the embryos were randomly selected and transferred into a sterile, flat bottomed 12-well tissue culture plate. Each well contained 10 ml of sterile seawater at 18° C. and an individual ascidian larva.

Larval settlement experiment: Three sound treatments were used: High and Low intensity vessel noise, and a Silent control. For each sound treatment, a water bath was used to maintain a constant water temperature at 18° C. (±1° C.) throughout. Each water bath contained a single 12-well tissue culture plate which was visually and acoustically transparent. The water baths were covered with shade cloth, providing a constant low light level, thereby eliminating interfering external light cues. Sponge rubber mats were placed under the water baths to prevent any transfer of acoustic energy from the surrounding environment into the experimental treatments. Prior to the commencement of any experiments, the absence of acoustical interference in the treatment baths was confirmed by recording from each water bath using a calibrated hydrophone. Sound treatments in each water bath were achieved by placing a loud speaker in the bottom, sealed within a waterproof plastic bag and held down by a lead weight. The speakers were connected to a MP3 player which continuously replayed a 4 minute sequence of the vessel noise recording. The volume control on the MP3 player was used to adjust the sound intensity in the tank to 126 dB and 100 dB re 1 μPa RMS for the High and Low intensity treatments respectively. The sound intensity was also monitored over the 100-10,000 Hz frequency range using a calibrated hydrophone. The experiment began at 1130 h and every 2 h from this time each tissue culture plate was removed from a water bath and examined under a binocular microscope (×40) to observe the status of each larva and classified as: swimming; immobile (larvae motionless when stimulated by gentle suction from the tip of a 200 μm pipette, larvae still coloured/opaque and body still intact); attached (larvae attached to the surface of the well or the meniscus of the water by head, remains attached when gently stimulated by water movement); metamorphic stage 1 (tail at right angles to head, tail beginning to turn transparent and starting to reabsorb, head darkening/pinkening, firmly attached to surface of well or meniscus); metamorphic stage 2 (tail reabsorption complete, pink colouration in head, larvae lobed shaped, stalk starting to appear); or dead (larvae transparent or emaciated, head and tail starting to fragment and shrink, no movement). The experiment was terminated when all experimental larva had either attached (and/or metamorphosed) or were dead.

### Results

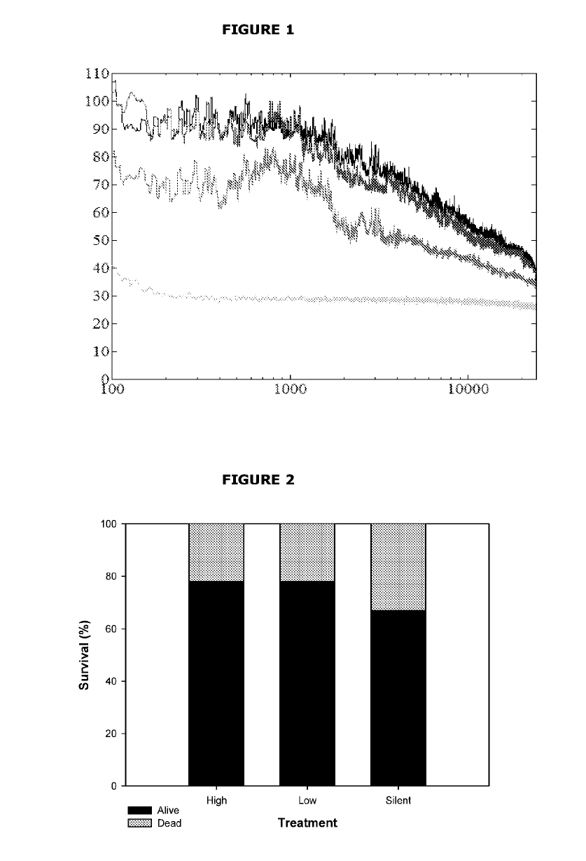

Vessel noise recordings: The vessel noise recorded from the passenger ferry was composed of predominantly lower frequency noise, between 100 and 1000 Hz and was measured to 126 dB re 1 μPa RMS at the source. For the High intensity treatments the experimental vessel noise replayed in the water baths was confirmed at 126 dB, and 100 dB for the Low intensity treatment. The replayed experimental noise had similar sound spectral composition to the noise recorded from the vessel in port. There was no external sound transfer influencing the Silent treatment, as confirmed by a sound recording from the Silent treatment water bath, with a mostly flat lined response at approximately 35 dB re 1 μPa, which also represented the lower recording limit of the recording equipment. FIG. 1 is a spectrogram of vessel noise when recorded; from the vessel berthed in port—top line, in High intensity treatment—second line, and in Low intensity treatment—third line, and Silent treatment, i.e., no vessel noise—bottom line.

Larval settlement experiment: The experiment ran over 28 hours by which time all surviving individual larvae in all treatments had settled and metamorphosed or were dead. There was no significant difference in larval survival among the twelve replicates within each of the treatments. Therefore, the larval survival data for the twelve replicates within each treatment were pooled to test for an overall treatment effect. There was no significant difference in larval survival among the three treatments, with 78% survival in the High and Low intensity vessel noise treatments and 67% in the Silent treatment. FIG. 2 is a bar graph showing the percentage mean survival for each treatment at the conclusion of the experiment.

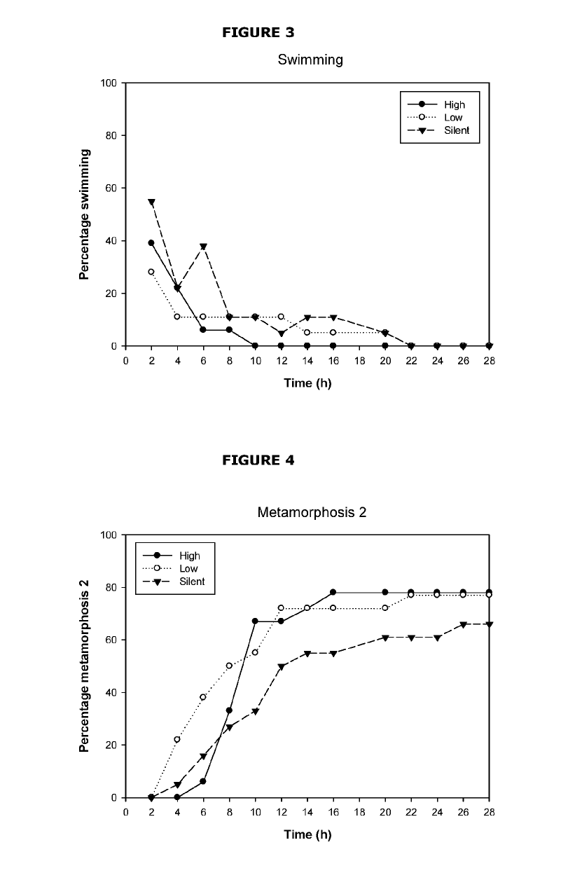

The results indicate that there was a faster reduction in the number of swimming larvae over time in the High intensity treatment. FIG. 3 shows the percentage of total number of ascidian larvae swimming over time (h). At the beginning of the experiment, 100% of the larvae were swimming when introduced into the experimental chambers. Within the first 2 hours, 40% had settled and by 10 hours, all of the larvae had ceased swimming in the High intensity treatment. Although there was a rapid initial settlement in the Low intensity and Silent treatments of approximately 30% and 50% respectively, it took 22 hours for all of the larvae to settle in both these treatments. This settlement pattern was reflected in numbers of larvae which were classed as ‘attached’, which significantly increased over the initial 5 hours in the High intensity treatment and then metamorphosed.

FIG. 4 shows the percentage of total number of ascidian larvae which have metamorphosed to stage 2 (as defined above). In both the Low and High intensity treatments, approximately 80% of larvae had settled and undergone metamorphosis (to stage 2) by 10-16 hours. In contrast, in the Silent treatment, only 60% larvae actually succeeded in developing to M2, and it took longer to get to this developmental stage. In the High intensity treatment, the majority of the larvae (approx. 60%) settled within a short time frame, showing exponential metamorphosis to stage 2 between 6 and 10 hours post hatch. In the Low intensity treatment, it took approximately 10 hours for 60% to undergo metamorphosis to stage 2, and in the Silent treatment, it took the entire 28 hours of the experiment. Metamorphosis was also more variable in the Silent treatment, with groups of larvae settling intermittently. Metamorphosis was more consistent and was exponential between 2 and 12 hours post hatch in the Low and High intensity treatments. Larvae in the Low intensity treatment started to undergo M2 sooner than the High intensity treatment, but by 8-12 hours, the numbers which metamorphosed were not significant different to the High intensity treatment.

The results demonstrate that settlement and metamorphosis in ascidian larvae is strongly influenced by vessel sound. Larvae exposed to High intensity vessel sound settled and metamorphosed significantly faster than larvae which were not exposed to any noise cues. Approximately 90% of the larvae from the treatments exposed to vessel noise had settled 6 hours after the commencement of the experiment. Development to M2 was achieved in approximately 80% of the larvae exposed to the sound treatments, compared with only 60% in the silent treatment. Over the duration of the experiment, there was exponential metamorphosis in larvae exposed to the High intensity treatment, and overall, larvae in the sound treatments demonstrated quicker metamorphosis, particularly during the first 10 hours of the experiment. There was no significant difference in larval survival between High, Low and Silent treatments indicating that the sound was primarily influencing settlement and metamorphosis behaviour and not the overall viability of the larvae.

### Example 2

Using abundance data from pre-soaked settlement panels and underwater loudspeaker (transducer) systems, differences were analyzed in individual organism fouling abundances between two treatments, Sound and Silent, with the Sound treatment replaying pre-recorded noise generated from a vessel in port (Straitsman, 125-m long, passenger vessel). Pre-soaked settlement panels were attached to three underwater loud speaker systems for the Sound treatments and three dummy speaker systems for the Silent treatments, and deployed at dispersed locations along a 0.5 km wharf in Bon Accord Harbour Kawau Island, New Zealand. Panels were arranged in two different orientations, substrate (downward) orientated and surface (upward) orientated to test for differences due to orientation, these orientations occurred together on a speaker as speaker systems were the limiting factor. Treatments were deployed by divers in the correct orientation at locations along the wharf whereby minimum acoustic overlap occurred. For 27 days (approximately an entire lunar cycle) the underwater loudspeakers in the Sound treatments were continuously broadcasting pre-recorded in port vessel noise (at 128 dB re 1 μPa RMS level in the 20-10000 Hz range), which was confirmed using a calibrated hydrophone and recorder, and the Silent treatment dummy system was left silent. The sound broadcast in the Sound treatments had a similar overall spectral composition to the source signals recorded from the vessel in port, with low frequencies in the range of 20-2000 Hz dominating. The Silent treatments had little to no sound transfer from the Sound treatments, with only ambient underwater sounds from the harbor present. At the conclusion of the experiment, divers collected the loud speaker systems, removed the settlement panels and placed them in individual sealed plastic bags to reduce loss of any organisms when transporting them to the laboratory for analysis. Analysis of the panels involved dividing each panel surface into 12 equal parts and counting and identifying to species level where possible all sessile fouling organisms with a 40× magnification under a dissecting microscope. Initially visual inspection of the Sound vs. Silent settlement panels and loudspeaker housing revealed much greater fouling in the Sound treatment. It also revealed there were differences in fouling abundance between the surface orientated and substrate orientated settlement panels.

The settlement panels in the Sound treatment orientated towards the substrate had a higher number of total organisms than in the Silent treatment of the same orientation, with the sound panels having a total of 2190 individuals as opposed with 756 individuals on the Silent panels. This was similar in the surface orientated panels, with the Sound treatment having a total of 397 individuals compared with 133 individuals in the Silent treatment.

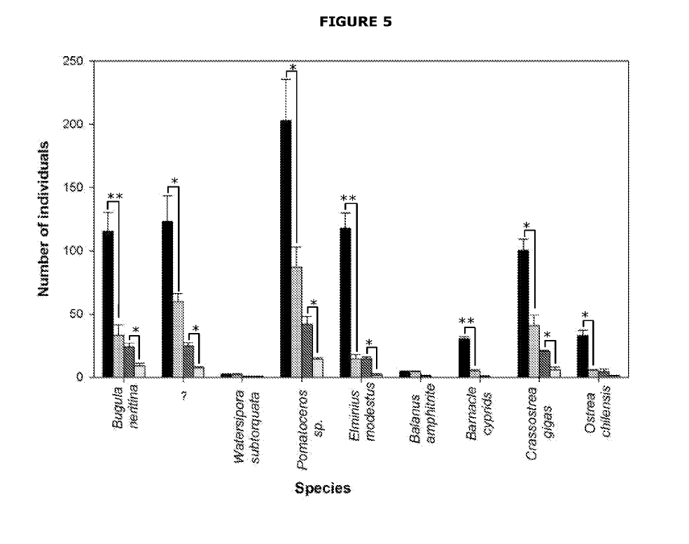

Overall, eight common fouling species were found attached to the settlement panels; bryozoans, Bugula neritina (erect branching), an unidentified grey bryozoan (encrusting), Watersipora subtorquata (encrusting), oysters, Crassostrea gigas, Ostrea chilensis, calcareous tube worm, Pomatoceros sp., barnacles, Elminius modestus, Balanus amphitrite, and also barnacle cyprids which were not identifiable to species. However, some species were more abundant than others in the both treatments. On the substrate orientated settlement panels in both the Sound and the Silent treatments all eight species were present, however, the tube worms (Pomatoceros sp.), erect and encrusting bryozoans, and the barnacle E. modestus dominated the Sound settlement panels, whereas only tube worms and encrusting bryozoans were dominant in the Silent controls. Using a individual t-test for the abundance of each species on the settlement plates there were significant differences detected in the mean number of individuals settling on the Sound versus the Silent treatments with higher number of organisms in the Sound treatment for B. neritina (tₜ₋ₜₑₛₜ=4.85, Pₜ₋ₜₑₛₜ=0.008), unidentified grey bryozoan (t=3.01, P=0.040), Pomatoceros sp. (t=3.21, P=0.030), E. modestus (t=8.21, P=0.001), unidentified barnacle cyprids (t=12.017, P=<0.001), C. gigas (t=5.60, P=0.005), and O. chilensis (t=6.27, P=0.003). FIG. 5 is a bar graph showing the mean (+S.E) number of individuals settled of each species for the Sound treatments; substrate orientated (black) and surface orientated (dark grey) and Silent treatments; substrate orientated (light grey) and surface orientated (white). Statistical results for t-tests, *<0.05, **<0.01, ***≦0.001. Using a Mann-Whitney Rank Sum Test, the size frequency of individuals within a species was also detected to be significantly different between the treatments, with significantly higher size of individuals in the Sound treatment compared to the Silent Treatment for B. neritina (U_{Mann-Whitney}=29452.0, P_{Mann-Whitney}=<0.001), unidentified grey bryozoan (U=43883.0, P=0.004), E. modestus (U=10312.0, P=0.013), and C. gigas (U=22531.0, P=0.001).

On the surface orientated settlement panels in the Sound treatments all eight species were present, however, the barnacle B. amphitrite and barnacle cyprids were absent and several other species had very low numbers in the Silent treatments. Again, the settlement panels were dominated by the tubeworms and bryozoans. Using a individual t-test for the abundance of each species on these settlement plates found there were significant differences in the mean number of individuals settling between the Sound and Silent treatments with higher number of organisms in the Sound treatments for B. neritina (tₜ₋ₜₑₛₜ=4.06, Pₜ₋ₜₑₛₜ=0.015), grey unidentified bryozoan (t=4.20, P=0.014), Pomatoceros sp. (t=4.42, P=0.011), E. modestus (t=6.89, P=0.002), and C. gigas (t=5.56, P=0.003).

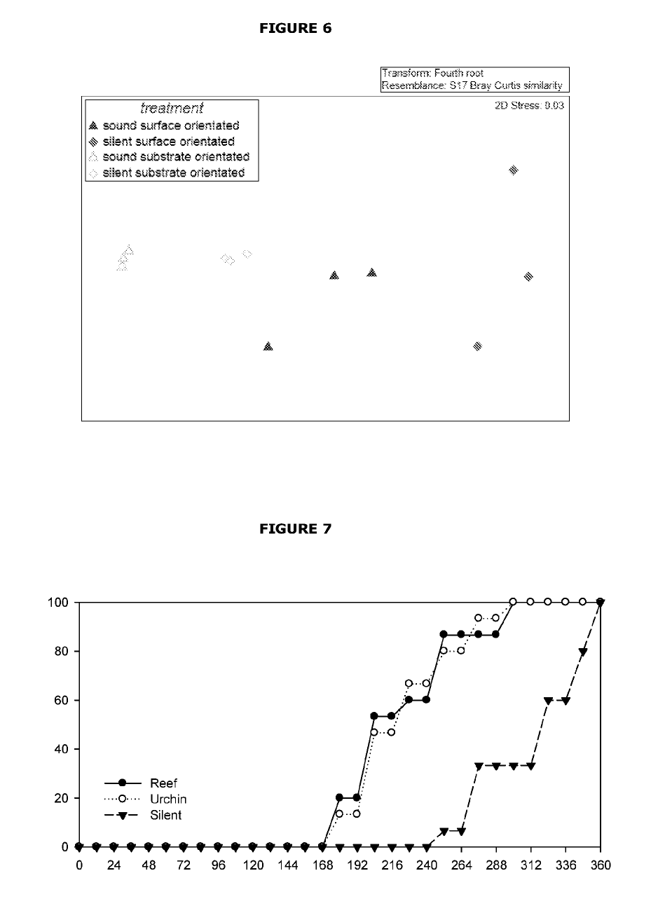

The ordination technique non-metric multidimensional scaling (MDS was used to examine relationships between mean number of individuals in each treatment and orientation. MDS creates low-dimensional maps of relationships among treatments and orientation, where the distance between points is proportional to their multivariate similarity. The analysis was run on a Bray-Curtis dissimilarity matrix derived from fourth-root transformed density data. Analysis of similarity (ANOSIM) was used to test for differences among these treatments and orientations. The mean number of individuals were grouped clearly in multivariate space according to treatment and treatment×orientation. FIG. 6 shows non-metric multidimensional scaling (MDS) analyses of total number of organisms attached to settlement panels in Sound and Silent treatments and with upward and downward orientations. Green hollow triangles represent Sound treatments, downward orientated, blue hollow diamonds represent Silent treatments, downward orientated, blue solid triangles represent Sound treatments, upward orientated, and red solid diamonds Silent treatments, upward orientated. All treatment and orientations showed strong grouping distinctions from each other (R_{ANOSIM}=0.796, P_{ANOSIM}=0.001.

Many of the species found attached to the settlement panels are known invasive species to New Zealand (e.g., B. neritina, W. subtorquata, C. gigas, and B. amphitrite). Most were thought to be introduced via vessel hull fouling, in ballast water and potentially due to aquaculture activities, and are considered as major fouling organisms in ports and harbours around New Zealand. Several of these species are known to cause large affects on native populations, for example, C. gigas is now a dominant structural component of fouling assemblages and intertidal shorelines in the northern harbours of New Zealand and the upper South Island. It is now the basis of New Zealand's oyster aquaculture industry after having displaced the native rock oyster, Saccostrea glomerata. The other species found on the settlement panels, native to New Zealand are known to have spread as marine fouling organisms to other countries (e.g., E. modestus and O. chilensis), vectors are thought to be largely via fouling on vessel hulls and larvae in ballast water.

### Example 3

This example investigates the effects of vessel noise of varying intensity on settlement of biofouling species in a marine environment and the settlement response of a common fouling ascidian species Ciona intestinalis.

### Methods

Vessel noise recording and processing: Vessel generator noises were recorded from a 25 m long steel-hulled fishing vessel berthed in the Port of Fremantle, Western Australia, in February 2012. Noise was recorded at four hull locations: (1) adjacent to the generator, (2) opposite generator, (3) stern, and (4) bow. At time of recording, no machinery other than the generator was operational, and no other vessels were operating in the vicinity.

A calibrated hydrophone (High Tech, Inc., Mississippi, USA, 129 HTI-96-Min) was used to record 5 min of continuous underwater noise emitted by the vessel generator. The hydrophone was placed approximately 50 cm from the hull and lowered 2 m into the water. The recording output was captured on a calibrated digital recorder.

Spectral plots were generated from the digital recordings, and an ANOVA was performed to determine if there was a significant difference (P<0.001) in noise intensity among the locations. The sub-samples were band pass filtered into four frequency bins: 30-100 Hz, 101-500 Hz, 501-2000 Hz, and 2001-20000 Hz and the overall mean proportion of total noise intensity was calculated for each frequency bin. For each location the proportion of total noise intensity was arcsine transformed and analysed using a Two-Way ANOVA, with Location and Frequency Bin as factors. Significant differences between proportions of total noise intensity were determined using the Holm-Sidak Test once the ANOVA had determined an overall significant difference among proportions (P<0.001).

In situ observations of level of fouling: Four 25 m fishing vessels of comparable hull design and antifouling treatment regime were berthed together at the time of this study. The location and type of generator was identical between the vessels. The level of biofouling present on each of the vessel hulls was estimated using in situ diver observations at four locations described above, and from examination of underwater video (Snake-Eye III TM156). All visual estimates of hull fouling were made by two divers independently using the Level of Fouling scale (developed by Floerl et al. (2005) Environ. Manage 35: 765-778). Each diver assessed a 2 m square area of the vertical side of each vessel at each location from the waterline to the top of the bilge keel.

Source of ascidian larvae: C. intestinalis adult specimens were collected from Lyttelton Harbour, New Zealand in January 2012. Eggs and sperm were removed using glass Pasteur pipettes, and the reproductive status was assessed visually prior to dissection to ensure only sexually mature individuals were used. Different donor specimens were used for cross fertilisation. The eggs were placed into a Petri dish containing 25 ml sterile seawater and ˜300 μl concentrated sperm and gently agitated to mix gametes. One hour after insemination, the seawater was changed to remove surplus sperm and the Petri dishes placed at 18-20° C. for 15-18 h to allow embryo development. Immediately prior to hatching, embryos were randomly selected and transferred to a sterile, flat bottomed 12-well tissue culture plate. Each well contained 10 ml sterile seawater at 18° C. and an individual C. intestinalis larva.

Larval settlement experiment: Larvae were exposed to a two minute noise recording from one of the four different locations on the vessel. Control larvae were exposed to no vessel noise. Water baths were used to maintain a constant water temperature of 18° C. (±1° C.). Each water bath contained a single 12-well tissue culture plate which was visually and acoustically transparent. The water baths were covered with shade cloth, providing a constant low light level, thereby reducing interference from external light cues. Foam rubber mats were placed under the water baths to prevent any transfer of acoustic energy from the surrounding environment into the experimental treatments. Prior to commencement of the experiment, the absence of acoustic interference in the treatment baths was confirmed by recording from each waterbath using a calibrated hydrophone.

Noise in each water bath was emitted from a speaker (Koninklijke Philips Electronics N.V., Netherlands, SBA1500, 4 Ohms; 100-18,000 Hz) submerged in the bottom of the water bath. The speakers were connected to an MP3 player which continuously replayed a 2 min sequence of the vessel noise recording. Three different two min sequences from each location were used to avoid pseudo-replication by using the same vessel recording for each replicate within the treatment. It was confirmed that the noise intensity of the noise replayed in the water baths for each recording was the same as that recorded at the corresponding vessel location. Recordings of the replayed generator noise were analysed and verified to have a similar spectral composition to the original recording of the noise from the vessel.

At two hourly intervals, the development of each larva was examined under a binocular microscope (×40). Larvae were classified according to their progressive stages in the settlement process: (1) Swimming; (2) Immobile (motionless when stimulated by gentle suction from the tip of a 200 μm pipette, still coloured/opaque, body intact); (3) Attached (attached to the surface of the well or meniscus of the water by head, remain attached when gently stimulated by water movement); (4) Metamorphic stage 1 (222 M1) (tail at right angles to head, tail beginning to turn transparent and starting to reabsorb, head darkening/starting to turn pink, firmly attached to surface of well or meniscus); (5) Metamorphic stage 2 (M2) (tail reabsorption complete, pink colouration in head, larva lobed shaped, stalk appeared); or (6) Dead (larvae transparent or emaciated, head and tail starting to fragment and shrink, no movement).

Data were examined to determine if there were any significant differences between replicates within treatments. No significant differences were found within treatments (controls or noise treatments) therefore all replicates within a treatment were pooled for analysis. Analyses for differences in larval, settlement, metamorphosis and survival were tested using Chi² analyses.

### Results

Vessel noise: The average noise intensity recorded at each vessel location was measured as follows: 140.6 dB re 1 μPa RMS at Location 1, 138.8 dB re 1 μPa at Location 2, 135.2 dB re 1 μPa at Location 3 and 127.5 dB re 1 μPa at Location 4. There was a significant difference in the noise intensity among all of the four locations (ANOVA; F=5349.4, P<0.001). There was also a significant difference among locations for frequency; Frequency Bin factor (F=30556.3, P<0.001), the Location factor (F=25.2, P<0.001) and in the interaction between Frequency Bin and Location (F=1437.9, P<0.001). All comparisons between Frequency Bin and Location showed a significant difference in proportion of total noise intensity (P<0.005) except for the comparison between Location 1 & 2, 2 & 3 and 1 & 4 in the highest frequency band 2001-20000 Hz. In general, the highest proportion of total noise intensity occurred in the 30-100 Hz frequency band, with the greatest of these occurring at Location 4 closest to the generator. The proportion of total noise intensity dropped as the frequency bands increased among all locations.

Level of vessel fouling: The level of fouling (LoF) was greatest at the location closest to the generator (Location 1) on all four vessels. Location 1 also had the highest intensity of noise. The biofouling level (relative abundance of fouling present on the vessel surface) decreased with increasing distance from the noise source (i.e. the generator), with the bow showing the least biofouling. All four vessels examined showed a similar trend, with the highest overall LoF at the generator and lowest at the bow. Intermediate LoF ranks were determined for the sites opposite the generator and at the stern. Biofouling consisted predominantly of colonial and solitary ascidians (Polycitor sp., Sigillina sp., Botrylloides sp., Styela sp.) bryozoans (Bugula sp., Zoobotryon verticillatum, Watersipora subtorquata, W. arcuata), serpulid polychaetes (unidentified) and porifera (unidentified). Although specific species data are not presented here, a total of 24 morphotypes were identified from the biofouling samples, of which four were confirmed to be non-indigenous species and three were cryptogenic.

Ciona intestinalis experiments: C. intestinalis larvae exposed to vessel generator noise from any of the four locations settled and metamorphosed significantly faster than control larvae not exposed to any generator noise. Approximately 50% of the surviving larvae that had been exposed to vessel generator noise from any one of the four locations had settled 6 hours after the commencement of the experiment, with the remaining larvae all settled by 18 hours. In contrast, for larvae not subjected to any vessel noise it took 15 h for 50% of surviving larvae to settle and a total of 26 hours for all surviving larvae to settle. Vessel noise also increased the rate at which larvae underwent metamorphosis. Development to M2 stage was achieved in 60% of the larvae exposed to the noise treatments (over a 12 h period), compared with only 20% in the control treatment over the same period. Larvae subjected to the two highest intensity noise treatments (immediately adjacent to the generator and opposite the generator) had a 100% survival rate compared to a maximum survival of only 66% for the silent control. There was no significant difference in larval settlement, metamorphosis or survival rates between the noise treatments from the different vessel locations.

The results demonstrate that broadcasting low frequency, high intensity sound increases settlement of biofouling organisms, and that the settlement and metamorphosis of ascidian larvae is strongly influenced by sound.

### Example 4

This example investigates the effects of noise of varying intensity and spectra on the development and survival of the settlement-stage pueruli of the southern spiny lobster, Jasus edwardsii.

### Methods

Source of pueruli: Natant pueruli of the southern spiny lobster, Jasus edwardsii were collected from beneath the wharf in the Port of Gisborne, or at Castlepoint on the east coast of North Island of New Zealand.

Sound recordings: Recordings of typical ambient underwater sound were made during the summer at dusk on a new moon at Waterfall Reef, a kelp-dominated rocky reef and at Nordic Reef, an urchin-dominated rocky reef both in north-eastern New Zealand. In situ habitat sounds were recorded in near calm conditions using a remote recording system which consisted of a calibrated hydrophone (see Example 3) connected to an automated recording system and a digital recorder Roland Edirol R09HR, contained in an underwater housing. No anthropogenic sources of noise, such as large ships or power boats, were present in the vicinity at the time of recording. Spectral composition of the digital recordings was analysed using MATLAB software with codes specifically written for these recordings. Ten typical 4 min sequences from each habitat recording were selected, and from these, three sequences were randomly selected and each transferred to MP3 player and used for playback in one of the three replicates for each sound treatment in the laboratory-based experiments.

Laboratory-based experiments: The experiment consisted of three sound treatments—a silent control and the two rocky reef habitat sounds. For each sound treatment, three replicate water baths were used to maintain a constant water temperature throughout the experiment (17° C.). The water baths were acoustically isolated using rubber mats and were kept under natural light. All replicates had a waterproofed weighted Phillips loudspeaker (4Ω, 5 W) submerged in the water bath.

For the sound replicates only, a DSE MP3 player was connected to the speaker and used to continually play a 4 min loop of recorded ambient underwater reef sound into the water bath and through the five replicate acoustically transparent 750 ml plastic containers each holding a single randomly assigned puerulus in filtered, UV-treated seawater together with a 200×90 mm piece of plastic mesh acting as a chemically inert settlement surface. A calibrated hydrophone and recorder (High Tech, Inc., Mississippi, USA HTI-96-MIN, Sound Devices, LLC., Wisconsin, USA 722 recorder) was used to adjust the sound in each tank to a level equivalent to the sound level of the natural habitat as recorded in the field. The replayed sounds in the experimental tanks were recorded for comparison with the source signals recorded from the natural habitats and to confirm the absence of significant sound in the silent treatments.

Pueruli were observed every 12 hours following initiation of the sound recording to determine whether an individual puerulus had moulted to the first instar juvenile stage. The time from initiation to the observation of a first instar juvenile was termed the time to moulting (TTM). The experiment was terminated when all pueruli in all treatments had moulted. At the first observation of moulting, juvenile pueruli were removed and immediately frozen.

Biochemical analyses: Lipid content of individual puerulus was measured gravimetrically using a modified Bligh and Dyer (1959) one-phase methanol/chloroform/water extraction (Jeffs et al., 2004, Comp Biochem Phys B 137:487-507) from individual lyophilised pueruli. After lipid extraction, individual puerulus were lyophilised again and homogenised with a micropestle. Protein content of individual pueruli was measured using the bicinchoninic acid (BCA) method using a Micro BCA protein assay kit (Thermo Scientific Pierce), using bovine serum albumin as the reference protein. A pre-weighed aliquot of the lyophilised samples from pueruli were digested for 12 h in 0.1 m NaOH at 50° C. to release bound protein. Total protein content of the pueruli was calculated as a percentage of original dry weight.

Data analyses: The non-parametric Kruskal-Wallis comparison of ranks was used to test for a difference in the median TTMs among the replicates within the same treatment (i.e., each treatment analysed separately) (Zar, Biostatistical Analysis, Fourth Edition ed. New Jersey: Prentice-Hall Inc. (1999)). No difference was found among the three replicates within a treatment, therefore, the data from the three replicates were pooled for an experiment-wide comparison using a Kruskal-Wallis test. The Kruskal-Wallis test was used to compare the distribution of median TTMs for pueruli among the treatments using the data pooled from the three treatments. For all statistical tests, P values ≦0.05 were considered significant. To isolate differences among individual treatments, a Tukey's Test pairwise multiple comparison procedure was used. A moulting rate for each treatment was also calculated with a Sen's slope analysis for the data points between the last sampling event prior to the first puerulus moulting and the sampling event when the last puerulus moulted.

Analyses of biochemical data: The non-parametric Kruskal-Wallis comparison of ranks was used to test for a difference in the total lipid and protein as a percentage of dry weight among treatments because the data were not from a normal distribution (Zar, Biostatistical Analysis, Fourth Edition ed. New Jersey: Prentice-Hall Inc. (1999)). To isolate differences among individual treatments a Tukey's Test pairwise multiple comparison procedure was used. All analyses were performed using the software Sigma Stat 4.0 (Systat Software, Inc.) and Minitab 16.1.0 (Minitab, Pty.).

### Results

Sound analyses: The field recording of the kelp-dominated rocky reef habitat had a peak in the spectra around 200-10,000 Hz, which is produced by the high frequency snaps of snapping shrimp. The urchin-dominated rocky reef recording had a peak in the spectra around 600-1500 Hz, which is produced by the feeding of the sea urchin, Evechinus chioroticus. The sound intensity was 109 and 116 dB re 1 μPa RMS level in the 100-24000 Hz for the kelp-dominated and urchin-dominated rocky reef treatments, respectively. The broadcast sound within the experimental tanks was reasonably consistent with the overall spectral composition and sound level to the source sound recorded from the natural habitats in situ, with a small reduction in sound level in the middle and higher frequencies (i.e., 800-2000 and 7000-20,000 Hz).

Pueruli time to moulting: Pueruli subjected to kelp-dominated rocky reef sound treatment had the shortest median TTM of 192 hours, followed by 216 hours for urchin-dominated rocky reef treatment, and 306 hours for silent treatment as shown in FIG. 7. Overall, the UM of pueruli was reduced by 38% in the presence of sound from kelp-dominated reef habitat and 30% in the presence of sound from urchin-dominated rocky reef habitat when compared to the silent (control) treatment.

Time to the first puerulus to complete moulting was 168 hours±8 S.E for both the kelp-dominated rocky reef and urchin-dominated rocky reef treatments. In contrast, time to first puerulus completing moulting in the silent treatment occurred after 240 hours±7.2 S.E. The time for all pueruli in each treatment to complete moulting for the kelp-dominated rocky reef and urchin-dominated rocky reef treatments was 288 hours±16 S.E. and 288 hours±21 S.E., respectively, compared with 348 hours±4 S.E for the silent treatment.

Biochemical analyses: For all treatments, both the lipid and protein content of first instar juvenile lobsters tended to decrease with increasing UM. Pueruli in the kelp-dominated rocky reef treatment had significantly more lipid (7.7% of dry weight) than either the urchin-dominated rocky reef (6.2%) or the silent treatment (6.1%) (Tukey's Test, P=<0.05). There was no significant difference in pueruli lipid content between the urchin-dominated rocky reef and silent treatments.

Pueruli in the kelp-dominated rocky reef treatment and urchin-dominated rocky reef treatments both had significantly more protein (35.7% and 34.4% of dry weight, respectively) than the silent treatment (30.5%) (Tukey's Test, P=<0.05). There was no significant difference in pueruli protein content between the kelp-dominated rocky reef and urchin-dominated rocky reef treatments (P>0.05).

The puerulus stage of spiny lobsters is lecithotrophic, relying solely on endogenous energy reserves accumulated during the extensive preceding phyllosoma phase, and consisting mostly of lipid and protein (Jeffs et al., 2001 Comp Biochem Phys A 129:305-311; Jeffs et al., 1999 Comp Biochem Phys A 123:351-357). There is evidence that delayed settlement leads to depletion of these reserves, which compromises subsequent survival (Fitzgibbon et. al, 2013, Fish In Press; Jeffs et al., 2001 Comp Biochem Phys A 129:305-31; Wilkin and Jeffs, 2011 Limno IOceanogr: Fluids & Environments 1:163-175). These results demonstrate that the development and survival of pueruli of the southern spiny lobster, Jasus edwardsii is strongly influenced by sound.

1. A method of reducing biofouling of a hull or part thereof or any submerged part of a vessel, or of a submerged structure or submerged part of a structure, or a submerged body, which comprises broadcasting sound into the marine environment in the vicinity of or at the hull, structure, or body but spaced therefrom at a frequency or in a frequency range and/or at a sound intensity and/or which varies and which is effective to attract one or more biofouling species to a marine-submersible or submerged sacrificial element associated with the sound source.

2-4. (canceled)

5. A system or apparatus for reducing biofouling, which comprises a marine-submersible or submerged sound transducer, a system for driving the transducer to broadcast sound into a marine environment at a frequency or in a frequency range and/or at a sound intensity and/or which varies and which is effective to attract one or more biofouling species to the submerged sound source, and a marine-submersible or submerged sacrificial element associated with the transducer providing a substrate to which biofouling species may attach.

6. A method according to claim 1 wherein the frequency range of the broadcast sound is in or predominantly in range 40 to 1200 Hz.

7. (canceled)

8. A method according to claim 1 wherein the broadcast sound comprises a recording made from a submerged microphone of real world sound from at least one vessel and/or in a port or natural reef environment.

9. (canceled)

10. A method according to claim 1 wherein the sound is broadcast in a direction away from the water surface.

11. A method according to claim 1 wherein the sound is broadcast at an intensity of at least 100 dB re 1 μPa at 1 m at the source.

12. A method for inducing settlement of settlement stages of marine species desired as seed for subsequent culture for aquaculture which comprises broadcasting sound into a marine environment or culture vessel in the vicinity of settlement material that can be recovered together with the seed for aquaculture, at a frequency or in a frequency range and/or at a sound intensity and/or which varies and which is effective to attract one or more of the desired aquaculture species to the submerged sound source and settlement material.

13. A method of claim 12 wherein the method further comprises promoting the growth and retention and survival of the settlement stages of marine species.

14. A method of claim 12 comprising the step of submerging settlement material specifically adapted for the attachment of settlement stages of desired marine species.

15-16. (canceled)

17. A method of claim 12 further comprising the step of
(a) processing the settlement material to recover the seed, or
(b) processing the settlement material once the attached larvae or spores of desired marine species have reached a desired size to recover the marine species.

18. (canceled)

19. A method of claim 12 wherein a submersible or submerged sound transducer, a system for driving the transducer is used to broadcast the sound into the marine environment or culture vessel, and wherein a submersible or submerged settlement material associated with the transducer provides a substrate to which the settlement stages of marine species may attach.

20. (canceled)

21. A method according to claim 12 wherein the frequency range of the broadcast sound is in or predominantly in range 40 to 1200 Hz.

22-23. (canceled)

24. A method according to claim 12 wherein the broadcast sound comprises a recording made from a submerged microphone of real world sound from at least one vessel and/or in a port or natural reef environment.

25. (canceled)

26. A method according to claim 12 wherein the sound is broadcast in a direction away from the water surface.

27. A method according to claim 12 wherein the sound is broadcast at an intensity of at least 100 dB re 1 μPa at 1 m at the source.

28. A method according to claim 12 wherein the marine species is selected from bivalves, crustaceans, and algae.
